(19) United States
(12) Patent Application Publication
(10) Pub. No.: US 2020/0397558 A1
(43) Pub. Date: Dec. 24, 2020

(54) REMOVABLE URETERAL STENTS AND METHODS OF USE OF THE SAME

(71) Applicant: Baylor College of Medicine, Houston, TX (US)

(72) Inventors: Chester J. Koh, Bellaire, TX (US); Eric Yin, Arcadia, CA (US); Valerie Pinillos, Houston, TX (US); Jun Xin Chen, New York, NY (US); Margaret Watkins, Houston, TX (US); Allen Zhao, Carroltton, TX (US)

(21) Appl. No.: 16/090,573

(22) PCT Filed: Apr. 5, 2017
(86) PCT No.: PCT/US17/26105

§ 371 Date: Oct. 1, 2018

Related U.S. Application Data
(60) Provisional application No. 62/320,974, filed on Apr. 11, 2016.

(51) Int. Cl. A61F 2/04; A61F 2/95

(52) U.S. Cl. CPC A61F 2/04; A61F 2/95; A61F 2002/048; A61F 2230/0091; A61F 2210/0014; A61F 2210/009; A61F 2230/0041; A61F 2002/9534

(57) ABSTRACT

One aspect of the invention provides a removable ureteral stent including: an elongated stent with opposing curls at either end; a coil of memory material attached to a curl at one end of the stent that can uncoil and elongate under tensile force, and does so elongate and uncoil at a lower tensile force than that needed to induce substantial uncoiling of either curl, and wherein the coil is at least long enough such that its distal end (from the end attached to the stent) can pass through an internal urethral sphincter, when the stent is in place in the ureter; and a magnet attached to the distal end of the coil.

### CROSS-REFERENCE TO RELATED APPLICATION

This application claims priority to U.S. Provisional Patent Application Ser. No. 62/320,974, filed Apr. 11, 2016. The entire content of this application is hereby incorporated by reference herein.

### BACKGROUND

The features of the stent lumen in U.S. Pat. No. 4,790,809 (Kuntz), including the multiple extendible and additional sections therein, are hereby incorporated by reference.

U.S. Pat. No. 4,790,809 (Kuntz), the ureteral stent therein has opposing curls at each end, which are effective in preventing stent migration and expulsion. Such stents are widely accepted because they can be easily introduced either endoscopically or during open surgery.

Most commercially available stents are difficult to remove. The usual method for removal of an indwelling stent from the ureter is a multi-step procedure requiring insertion of removal instruments such as a cystoscope into the subject's urethra, which is so potentially painful that it can require a general anesthetic.

### SUMMARY

One aspect of the invention provides a removable ureteral stent including: an elongated stent with opposing curls at either end; a coil of memory material attached to a curl at one end of the stent that can uncoil and elongate under tensile force, and does so elongate and uncoil at a lower tensile force than that needed to induce substantial uncoiling of either curl, and wherein the coil is at least long enough such that its distal end (from the end attached to the stent) can pass through an internal urethral sphincter, when the stent is in place in the ureter; and a magnet attached to the distal end of the coil.

This aspect of the invention can have a variety embodiments. The memory material can be a wire or a flattened ribbon shape.

The magnet can be a ferromagnet. The ferromagnet can be selected from the group consisting of: a rare earth magnet and an AlNiCo magnet. The ferromagnet can include a member selected from the group consisting of: Nd₂Fe₁₄B (neodymium), SmCo₅, SmCo₇, SmFe₇, SmCu₇ and SmZr₇.

One or more selected from the group consisting of: the magnet, the coil, or the stent can be coated with a biocompatible material. One or more selected from the group consisting of: the magnet, the coil, or the stent can be coated with a material selected from the group consisting of poly(p-xylylene) polymer and Ni—Cu—Ni alloy.

The curls can be substantially J-shaped. The curls can curl through at least ¾ of one completed circle.

The coil can include nitinol. The coil can be at least long enough such that its distal end (from the end attached to the stent) can pass through a urethral opening when the stent is in place in the ureter.

Another embodiment of the invention provides a method of removing a ureteral stent from a mammalian subject, wherein the ureteral stent includes an elongated stent with opposing curls at either end, a coil of memory material attached at one end of the stent that can uncoil and elongate under tensile force, and does so elongate and uncoil at a lower tensile force than that needed to induce substantial uncoiling of either curl, and a magnet attached to the distal end of the coil. The method includes: positioning an external magnetic force proximal to the pubic area of the mammalian subject; drawing the external magnetic force towards the mammalian subject's urethral opening, thereby drawing the magnet down the mammalian subject's urethra and towards the urethral opening; and pulling the magnet or the coil, which in turn pulls the stent through the urethral opening to remove it.

This aspect of the invention can have a variety embodiments. The first end of the ureteral stent can be positioned in the bladder of the mammalian subject and the second end of the ureteral stent is positioned in the ureter or kidney of the mammalian subject. The pulling can be by grabbing the magnet or coil with pliers or forceps, and then pulling using the pliers or forceps.

The external magnetic force can be applied using a paramagnet or an electromagnet. The pulling force can be strong enough to partially uncoil the curls.

The magnet can be withdrawn to a position in the urethra at which it can be grasped or hooked.

Another aspect of the invention provides a kit including: a removable ureteral stent and instructions to perform a method as described herein. The removable ureteral stent includes: an elongated stent with opposing curls at either end; a coil of memory material attached to the curl at one end of the stent that can uncoil and elongate under tensile force, and does so elongate and uncoil at a lower tensile force than that needed to induce substantial uncoiling of either curl, and wherein the coil is at least long enough such that its distal end (from the end attached to the stent) can pass through the internal urethral sphincter, when the stent is in place in the ureter; and a magnet attached to the distal end of the coil.

### BRIEF DESCRIPTION OF THE DRAWINGS

FIG. 1A depicts a first embodiment of a removable stent in place in a ureter.

FIG. 1B depicts a partial cross-section of another embodiment of a removable stent.

FIG. 2 depicts the embodiment of FIG. 1 acted on by an external magnet.

FIG. 3A is an elevational view of a ribbon-like coil, attached to a magnet.

FIG. 3B is a plan view of the coil of FIG. 3A.

FIG. 3C is an elevational view of a wire coil, attached to a magnet.

### DETAILED DESCRIPTION

Unless defined otherwise, all technical and medical terms used herein have the same meaning as commonly understood by one of ordinary skill in the art to which this invention belongs.

As used herein, the singular form “a,” “an,” and “the” include plural references unless the context clearly dictates otherwise.

As used herein, the term “alloy” refers to a mixture or metallic solid solution composed of two or more elements.

As used in the specification and claims, the terms “comprises,” “comprising,” “containing,” “having,” and the like have the meaning ascribed to them in patent law and can mean “includes,” “including,” and the like.

Unless specifically stated or obvious from context, the term “or,” as used herein, is understood to be inclusive or alternative.

As used herein, the term “mammalian subject,” includes, but is not limited to, humans (i.e., a male or female of any age group, e.g., a pediatric subject (e.g., infant, child, adolescent) or adult subject (e.g., young adult, middle aged adult or senior adult)) and/or other primates (e.g., cynomolgus monkeys, rhesus monkeys), mammals, including commercially relevant mammals such as cattle, pigs, horses, sheep, goats, cats, and/or dogs; and/or birds, including commercially relevant birds such as chickens, ducks, geese, quail, and/or turkeys

The term “magnet” is defined as paramagnet or a material which can be magnetized or attracted by a magnetic force.

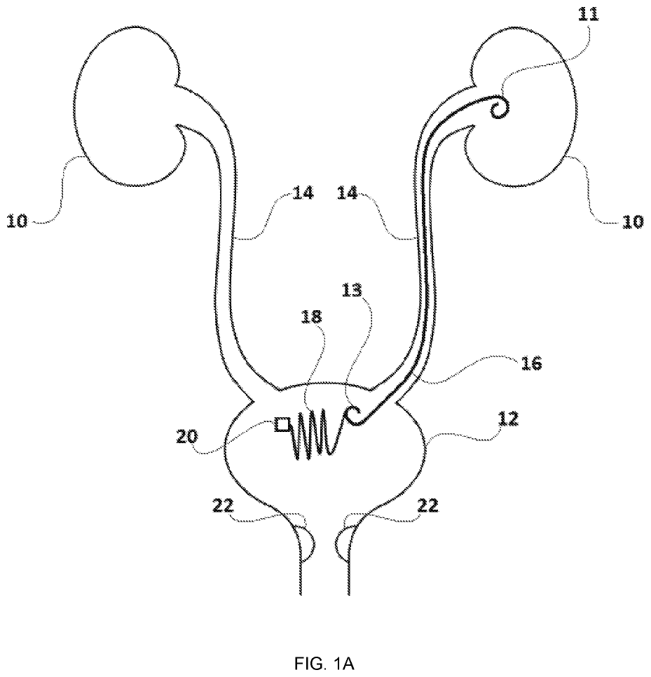

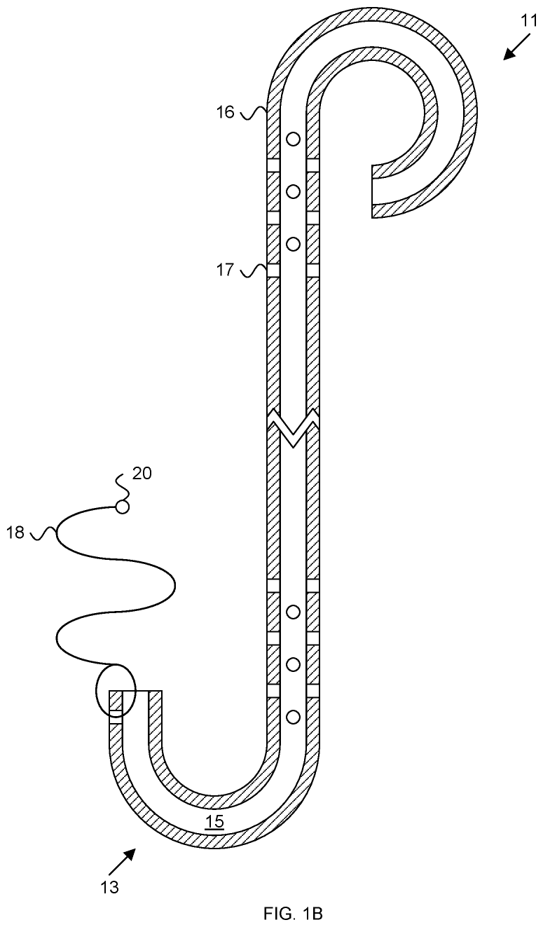

Referring to FIGS. 1A & 2, a stent 16 (which may be made of polymers or a number of materials), with opposing curls 11, 13 at its ends, is shown in place in ureter 14. One end and curl 13 of stent 16 lies in bladder 12 and the opposing end and curl 11 lies in kidney 10. The curls 11 and 13 assist in keeping stent 16 in place in ureter 14. The curls 11 and 13 can be J-shaped (e.g., as shown at curl 13 in FIG. 1B) or more curled than the curls 11 and 13 shown, and preferred embodiments may be curled to define less than one complete circle, about one complete circle, exactly one complete circle, or more than one complete circle from a side view. Some exemplary curls in a stent are illustrated in U.S. Pat. No. 5,599,291 (Balbierz), U.S. Pat. No. 6,887,215 (McWeeney), U.S. Pat. No. 8,252,065 (Ward), U.S. Pat. No. 9,308,359 (Ward), and U.S. Pat. No. 9,445,884 (Barron). A variety of ureteral stents are available from Boston Scientific Corporation of Marlborough, Mass., Cook Group, Inc. of Bloomington, Ind. and Olympus Corporation of the Americas of Center Valley, Pa.

Referring to FIG. 1B, stent 16 can define a central lumen 15 to facilitate drainage from one end to another end. Stent 16 can further define one or more side holes 17 through with fluid can drain into or emerge from a central lumen 15. Additionally or alternatively, the central lumen 15 can be open at one or more ends of the stent 16.

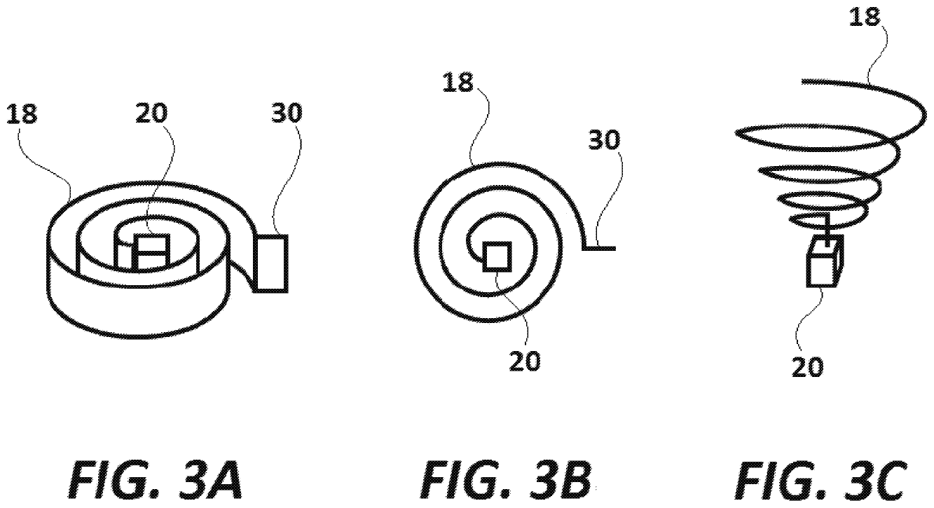

Coil material 18 is attached to the end of stent 16 residing in bladder 12, and magnet 20 is attached to the opposite end of coil material 18. As shown in FIGS. 3A-3C, coil material 18 can be a wire (as in FIG. 3C) or a ribbon shape (as in FIGS. 3A-3B). Where coil material 18 is a ribbon shape, it is preferred to angle one end 30 to make it easier to attach to stent 16. Coil material 18 can be a shape memory polymer or a memory material that assumes a coil shape, absent an elongating force. Coil material 18 may be made of or include nitinol, which has properties of shape memory and super-elasticity.

Magnet 20 can be iron, iron alloys, rare earth elements or alloys, an AlNiCo magnet, another ferrous material, and the like. The alloys can include one or more of: Nd₂Fe₁₄B (neodymium), SmCo₅, SmCo₇, SmFe₇, SmCu₇ and SmZr₇. Magnet 20 can be sized for passage through a subject's urethra. For example, the magnet 20 can have a maximum cross-sectional dimension (e.g., a diameter in circular cross-section, a diagonal in a square cross-section, and the like) along at least one face between about 2 mm and about 3 mm, between about 2 mm and about 4 mm, and the like.

Any of stent 16, coil material 18 and magnet 20 may be coated with a biocompatible material, including but not limited to PFTE or other biocompatible polymers, including poly(p-xylylene) polymer (commonly referred to as Parylene C) and Ni—Cu—Ni alloy.

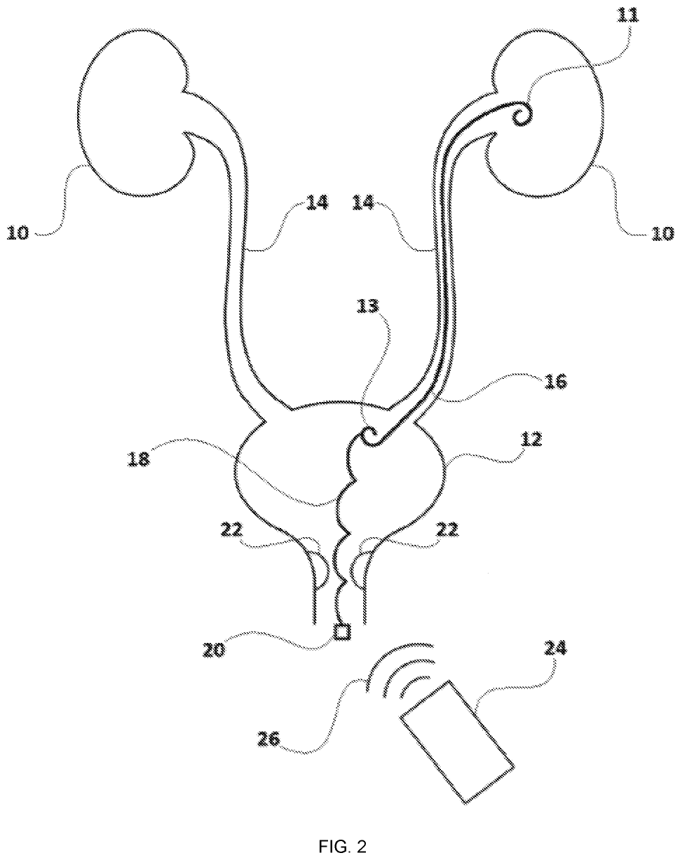

Referring to FIG. 2, external magnet 24 exerts attractive force 26 on magnet 20, thereby elongating coil material 18. External magnet 24 can be any type of paramagnet (similar to or different from magnet 20) or an electromagnet. Magnet 24 is moved such that force 26 brings magnet 20 through internal urethral sphincter 22 and into the urethra. Magnet 24 can be of sufficient strength to facilitate movement of at least the magnet 20 through the urethra but not so strong that it can cause damage or injury to the subject. For example, the magnet 24 can apply a magnetic force between about 0.001 mT to about 10 mT, between 0.1 mT to about 6 mT, between 1 mT to about 6 mT, about 5 mT, and the like.

Coil material 18 is preferably of sufficient length to allow magnet 20 past sphincter 22, prior to movement of stent 16 out of ureter 14. Exemplary lengths for coil material 18 include between about 2 cm and about 9 cm, between about 10 cm and about 20 cm, between about 20 cm and about 30 cm, and the like. Removal of stent 16 would be considerably more difficult if magnet 20 stayed on the bladder side of sphincter 22.

In one embodiment, the coil material 18 has a length reflecting the expected length between a subject's bladder 12 (e.g., proximal to a ureter 14) and urethral opening so that the magnet 20 can be withdrawn through the urethral opening before the stent 16 engages with the subject's urethra and/or before one or more curls 11, 13 unwind.

Complete or partial withdrawal of the magnet 20 from the subject's bladder 12 allows a medical professional to engage the magnet 20 and/or coil material 18 and remove the stent 16 using tactile feedback to detect if the stent 16 catches on the subject's anatomy.

More preferably, coil material 18 is of sufficient length to allow magnet 20 past the opening of the urethra, where it can be grabbed (e.g., with a grasping tool such as pliers or forceps) and pulled. The pulling force is preferably sufficient to cause some unwinding of the curls 11, 13 in stent 16. Then, stent 16 will easily be removed by such pulling force. Another alternative is to use force 26 to move magnet 20 into the urethra, where it is grasped or hooked using a catheter or stent inserted into the urethra and capable of grasping or hooking it. The inserted catheter or stent is then pulled out, thereby also removing stent 16.

Embodiments of the invention can removed from the subject's (e.g., a pediatric subject's) ureter without general anesthesia. Subject comfort can be measured using the Wong-Baker Faces scale. In one embodiment, removal of the stent 16 yields a Wong-Baker Faces rating of less than 4 (e.g., for an individual patient or as a mean, median, or mode across a population or sub-population).

Further aspects of the invention include kits including one or more components described herein and instructions for performing the methods as described herein.

The specific methods, procedures and apparatus described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicant.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A removable ureteral stent comprising:
an elongated stent with opposing curls at either end;
a coil of memory material attached to a curl at one end of the stent that can uncoil and elongate under tensile force, and does so elongate and uncoil at a lower tensile force than that needed to induce substantial uncoiling of either curl, and wherein the coil is at least long enough such that its distal end (from the end attached to the stent) can pass through an internal urethral sphincter, when the stent is in place in the ureter; and
a magnet attached to the distal end of the coil.

2. The removable ureteral stent of claim 1 wherein the memory material is a wire or a flattened ribbon shape.

3. The removable ureteral stent of claim 1 wherein the magnet is a ferromagnet.

4. The removable ureteral stent of claim 3 wherein the ferromagnet is selected from the group consisting of: a rare earth magnet and an AlNiCo magnet.

5. The removable ureteral stent of claim 3 wherein the ferromagnet includes a member selected from the group consisting of: Nd₂Fe₁₄B (neodymium), SmCo₅, SmCo₇, SmFe₇, SmCu₇ and SmZr₇.

6. The removable ureteral stent of claim 1 wherein one or more selected from the group consisting of: the magnet, the coil, or the stent is coated with a biocompatible material.

7. The removable ureteral stent of claim 6 wherein one or more selected from the group consisting of: the magnet, the coil, or the stent is coated with a material selected from the group consisting of poly(p-xylylene) polymer and Ni—Cu—Ni alloy.

8. The removable ureteral stent of claim 1 wherein the curls are substantially J-shaped.

9. The removable ureteral stent of claim 1 wherein the curls curl through at least ¾ of one completed circle.

10. The removable ureteral stent of claim 1 wherein the coil includes nitinol.

11. The removable ureteral stent of claim 1 wherein the coil is at least long enough such that its distal end (from the end attached to the stent) can pass through a urethral opening when the stent is in place in the ureter.

12. A method of removing a ureteral stent from a mammalian subject, wherein the ureteral stent includes an elongated stent with opposing curls at either end, a coil of memory material attached at one end of the stent and can uncoil and elongate under tensile force, and does so elongate and uncoil at a lower tensile force than that needed to induce substantial uncoiling of either curl, and a magnet attached to the distal end of the coil, the method comprising:
positioning an external magnetic force proximal to the pubic area of the mammalian subject;
drawing the external magnetic force towards the mammalian subject's urethral opening, thereby drawing the magnet down the mammalian subject's urethra and towards the urethral opening; and
pulling the magnet or the coil, which in turn pulls the stent through the urethral opening to remove it.

13. The method of claim 12 wherein the first end of the ureteral stent is positioned in the bladder of the mammalian subject and the second end of the ureteral stent is positioned in the ureter or kidney of the mammalian subject.

14. The method of claim 12 wherein the pulling is by grabbing the magnet or coil with pliers or forceps, and then pulling using the pliers or forceps.

15. The method of claim 12 wherein the external magnetic force is applied using a paramagnet or an electromagnet.

16. The method of claim 12 wherein the pulling force is strong enough to partially uncoil the curls.

17. The method of claim 12 wherein the magnet is withdrawn to a position in the urethra at which it can be grasped or hooked.

18. A kit comprising:
a removable ureteral stent comprising:
an elongated stent with opposing curls at either end;
a coil of memory material attached to the curl at one end of the stent that can uncoil and elongate under tensile force, and does so elongate and uncoil at a lower tensile force than that needed to induce substantial uncoiling of either curl, and wherein the coil is at least long enough such that its distal end (from the end attached to the stent) can pass through the internal urethral sphincter, when the stent is in place in the ureter; and
a magnet attached to the distal end of the coil; and
instructions to perform the method of claim 12.
